(19) United States
(12) Patent Application Publication
(10) Pub. No.: US 2008/0193576 A1
(43) Pub. Date: Aug. 14, 2008

(54) Antiphlogistic and analgesic composition for topical use in a region of an animal locomotive system

(76) Inventors: Vincenzo Massimo Lombardo, Milano (IT); Alberto Antonio Molinari, Milano (IT); Ruggero Ferraiolo, Milano (IT)

Correspondence Address: YOUNG & THOMPSON, 209 Madison Street, Suite 500, ALEXANDRIA, VA, 22314, US

(21) Appl. No.: 11/705,753

(22) Filed: Feb. 14, 2007

(51) Int. Cl. A61K 36/28

(52) U.S. Cl. 424/764

(57) ABSTRACT

An antiphlogistic and analgesic composition for topical use consists, by weight, of from about 0.01-3% hyaluronic acid, from about 0.5-7% arnica glycolic extract, from about 2-10% of any hydrogel-soluble, non-steroidal anti-inflammatory agent, and 70-95% hydrogel.

### BACKGROUND OF THE INVENTION

The present invention relates to an antiphlogistic and analgesic composition for use on an epidermis site where pains are felt from muscles, articulations, tendons, and ligaments caused by pathologies, contusions, and trauma.

### DESCRIPTION OF THE RELATED ART

The prior art comprises a number of compositions in the form of ointments, creams, sprays, or foams which are able to assuage the above-specified pains and to treat their cause to a notable or little extent. Examples are the commercial products sold under the names of Voltaren®, Algesal®, Lasonil®, Artrosilene®, wherein the active ingredients are, respectively, diclofenac diethyl ammonium, diethyl amine salicylate, ketoprofene, lysine salt ketoprofene. A number of other products are used for the same purposes that employ other active ingredients comprised in the pharmacopeia relevant to said purposes. JP 11 279 065 discloses an anti-inflammatory product in the form of a topical solution having two water-soluble, anti-inflammatory agents, one of the steroidal type and the other of the non-steroidal type, and hyaluronic acid.

The efficacy of the analgesic and anti-inflammatory effect of the known compositions is affected by the fact that such compositions comprise, in most cases, an active ingredient only which is not well vehiculated into the suffering body region. Consequently, the efficacy of said compositions may be short-lived and not so extended to the concerned region. Moreover, these known compositions may have undesired effects due to light or notable hyperaesthesia against one or more components and to allergic reactions.

### SUMMARY OF THE INVENTION

The inventive composition obviate the above-mentioned limitations and drawbacks and offer other advantages that will be described herein.

The inventive composition comprises as active ingredients a salt of hyaluronic acid, an arnica extract and a non-steroidal anti-inflammatory agent (FANS—as for instance methyl salicylate, dychlofenac, lysine salt ketoprofene) soluble in a gel and, as vehicle of the active ingredients, hydrogel of the type used in medicine for applying ultrasounds and electric transmissions, type of gel which proved to give the compositions the best physical properties and that will be referred to as—hydrogel—in the following.

Hyaluronic acid as an anti-inflammatory and chemotactic agent has been known for a long time and is generally and successfully used in injectable solutions to treat osteoarthrosis, gonarthrosis, wounds and burns, in cosmetic surgery and culture and also as a component for ophthalmic use. In the inventive composition, hyarulonic acid is preferably used as sodium hyaluronate, dissolved in just necessary purified water before preparing the composition.

The efficacy is known for a long time of: arnica in the treatment of consequences of trauma and of symptoms similar with “post-trauma”; FANS in the treatment of pains and as antiphlogistic agent; hydrogel as vehicle of medicaments applied to the epidermis.

The combination in the invented composition of said three active ingredients and the presence of a hydrogel, all the four in the proposed proportions, leads to a fluid and rubbable product which is absorbed rapidly by the epidermis and is of high efficacy in assuaging pains of articular and osteomuscular origin, in the reduction of possible inflammation and in the absorption of possible serous or blood extravasation caused by trauma or contusion.

Such an efficacy is substantially due to the anti-inflammatory and chemotactic properties as well as to the notable hydrophilia of the hyaluronic acid. The latter makes pores of the basal membrane dilate and, supported by the high vehiculation favoured by the hydrogel, causes itself and the other two antiphlogistic and analgesic components to rapidly penetrate into the suffering region. Moreover, undesirable effects and allergic reactions of this composition have not been recorded in case of long-term use.

### DESCRIPTION OF THE PREFERRED EMBODIMENTS

According to first formulae of this composition that proved to be of considerable efficacy through tests carried out on many persons, 100 g of the composition comprises by weight:

sodium hyaluronate, from about 0.01-3.00%,

arnica glycolic extract, from about 0.5-7.0%,
any hydrogel-soluble, non-steroidal anti-inflammatory agent, from about 2-10%,

hydrogel, from about 70-95%,

purified water, about 0.2%,

As it is known, there are a number of non-steroidal, anti-inflammatory agents, and the inventors proved that each, provided it is soluble in hydrogel, may be used in these compositions in a proportion of from about 2-10% in order to provide the composition with the desired efficacy. It has been shown that the most efficacious anti-inflammatory agent in the inventive composition is methyl salicylate.

According to a formula of this composition that proved to be the most efficacious through tests carried out on a great number of persons and in a number of percentage alternatives for the four components, the following formula in weight reaches the desired purposes:

sodium hyaluronate about 0.2%,

purified water about 0.2%,

arnica glycolic extract about 2%,

methyl salicylate about 5%,

hydrogel about 92.6%.

1. An antiphlogistic and analgesic composition for use in a region of an animal locomotive system comprising as active ingredients a non-steroidal, anti-inflammatory agent and hyaluronic acid and arnica mixed in a product adapted to vehiculate the active ingredients, wherein the composition is a mixture of a salt of hyaluronic acid, an arnica glycolic extract, and a hydrogel-soluble, non-steroidal anti-inflammatory agent and hydrogel as vehicle of the active ingredients.

2. A composition according to claim 1, comprising by weight:
sodium hyaluronate, from about 0.01-3.0%,
arnica glycolic extract, from about 0.5-7.0%,
any hydrogel-soluble, non-steroidal anti-inflammatory agent, from about 2-10%,
hydrogel, from about 70-95%, and
purified water, about 0.2%.

3. A composition according to claim 1, wherein the hydrogel-soluble, non-steroidal anti-inflammatory agent is methyl salicylate.

4. An antiphlogistic and analgesic composition for use in a region of an animal locomotive system comprising as active ingredients a non-steroidal, anti-inflammatory agent and hyaluronic acid and arnica mixed in a product adapted to vehiculate the active ingredients, wherein the composition consists by weight of:
sodium hyaluronate, from about 0.2%,
purified water, from about 0.2%,
arnica glycolic extract, from about 2%,
methyl salicylate, from about 5%, and
hydrogel, from about 92.6%.

5. A composition according to claim 2, wherein the hydrogel-soluble, non-steroidal anti-inflammatory agent is methyl salicylate.
